(19) United States
(12) Patent Application Publication
(10) Pub. No.: US 2012/0271147 A1
(43) Pub. Date: Oct. 25, 2012

(54) APPARATUS, METHOD, AND COMPUTER-ACCESSIBLE MEDIUM FOR B1-INSENSITIVE HIGH RESOLUTION 2D T1 MAPPING IN MAGNETIC RESONANCE IMAGING

(75) Inventors: Daniel Kim, Park City, UT (US); Riccardo Lattanzi, New York, NY (US); Christian Glaser, Muenchen (DE); Michael Recht, Scarsdale, NY (US)

(73) Assignee: New York University, New York, NY (US)

(21) Appl. No.: 13/453,365

(22) Filed: Apr. 23, 2012

Related U.S. Application Data
(60) Provisional application No. 61/478,271, filed on Apr. 22, 2011.

(51) Int. Cl. A61B 5/055

(52) U.S. Cl. 600/410

(57) ABSTRACT

Exemplary systems, methods and computer-accessible mediums can be provided for imaging at least one anatomical structure. For example, it is possible to direct a saturation-recovery (SR) pulse sequence having fast spin echo (FSE) to or at the anatomical structure(s). At least one T₁ image of the at least one anatomical structure can be generated based on the SR pulse sequence. In one example, the anatomical structure(s) can include a hip. According to another example, T₁ image(s) can include a plurality of T₁ images generated or provided in a plurality of rotating radial planes.

### CROSS-REFERENCE TO RELATED APPLICATION(S)

The present application relates to and claims priority from U.S. Provisional Patent Application No. 61/478,271 filed Apr. 22, 2011, the entire disclosure of which is incorporated herewith by reference.

### FIELD OF THE DISCLOSURE

The present disclosure relates to exemplary embodiments of apparatus, methods, and computer accessible-medium for medical imaging, and more particularly, to exemplary embodiments of apparatus, methods, and computer accessible-medium for longitudinal relaxation time (T₁) mapping using fast spin echo.

### BACKGROUND INFORMATION

It has been recognized that femoroacetabular impingement (FAI), a condition in which structural abnormalities of the femoral headneck junction and/or acetabulum cause mechanical blockage in the terminal range of hip motion, can lead to osteoarthritis (OA) of the hip (see, e.g., Ganz R, Parvizi J, Beck M, Leunig M, Notzli H, Siebenrock K A. Femoroacetabular impingement: a cause for osteoarthritis of the hip. Clinical Orthopaedics & Related Research 2003; 417:112-120; see also Wagner S, Hofstetter W, Chiquet M, Mainil-Varlet P, Stauffer E, Ganz R, Siebenrock K A. Early osteoarthritic changes of human femoral head cartilage subsequent to femoro-acetabular impingement. Osteoarthritis & Cartilage 2003; 11(7):508-518). In FAI, the abnormal contact between the acetabular rim and femoral neck can cause chondral and labral damage, which can progress over time and result in OA of the hip joint if the underlying cause of impingement is not addressed surgically (see, e.g., Tanzer M, Noiseux N. Osseous abnormalities and early osteoarthritis: the role of hip impingement. Clinical Orthopaedics & Related Research 2004; 429:170-177).

MR imaging has emerged as a diagnostic modality for suspected FAI due to its multiplanar image acquisition capability and its high soft tissue contrast. The acetabular cartilage's and labrum's position and orientation within the pelvis make MR imaging of these structures in three orthogonal planes susceptible to partial volume effects. One approach to minimize partial volume averaging can be to image the acetabular rim and cartilage in a set of rotating radial planes. Imaging in rotating radial planes can exploit the geometry of the hip joint and can allow orthogonal display of the whole acetabular rim around its circumference. This imaging technique has been shown to be potentially useful in identifying obliquely oriented tears in the anterosuperior and posterosuperior sections of the labrum.

Corrective surgical procedures aimed at removing the bony abnormalities of FAI and treating the associated labral and cartilage abnormalities are traditionally less likely to be successful in patients presenting with extensive articular cartilage injuries (see, e.g., R Beck M, Leunig M, Parvizi J, Boutier V, Wyss D, Ganz R. Anterior femoroacetabular impingement: part II. Midterm results of surgical treatment. Clinical Orthopaedics & Related Research 2004; 418:67-73), for whom viable treatment is traditionally arthroplasty. Therefore, it can be preferable to detect cartilage damage in its early stages. Cartilage that appears morphologically normal in routine MRI may already be irreversibly compromised in early OA. MR-based biochemical imaging techniques, such as delayed Gadolinium-Enhanced MRI of Cartilage (dGEMRIC) (see, e.g., Bashir A, Gray M L, Burstein D. Gd-DTPA2- as a measure of cartilage degradation. Magnetic Resonance in Medicine 1996; 36(5):665-673; see also Bashir A, Gray M L, Hartke J, Burstein D. Nondestructive imaging of human cartilage glycosaminoglycan concentration by MRI. Magnetic Resonance in Medicine 1999; 41(5):857-865), have been proposed as an early diagnostic tool for the evaluation of chondral lesions. In dGEMRIC, negatively charged contrast agent (e.g., Gd-DTPA2-) can typically be administered prior to an exercise protocol, in order to exploit the different Gd-DTPA kinetics between the healthy and compromised cartilage, and imaging is typically performed to measure delayed contrast enhancement of compromised cartilage, which reflects the local concentration of glycosaminoglycans (GAG) in an inverse relationship. The areas with depleted GAG generally have higher concentrations of Gd-DTPA2-, which can be reflected in the measured T₁, Therefore, dGEMRIC can provide an indirect visualization of GAG loss, which can be an early sign of cartilage degeneration (see, e.g., Kim Y J, Jaramillo D, Millis M B, Gray M L, Burstein D. Assessment of early osteoarthritis in hip dysplasia with delayed gadolinium-enhanced magnetic resonance imaging of cartilage, Journal of Bone & Joint Surgery—American Volume 2003; 85-A(10):1987-1992).

A fast 2-angle T₁ mapping (F2T1) pulse sequence based on three dimensional (3D) gradient echo readout has also been introduced and validated for dGEMRIC in the hip. The F2T1 pulse sequence can be more time-efficient than two-dimensional (2D) multi-point inversion recovery (IR) and saturation recovery (SR) pulse sequences, which can be problematic for clinical use due to their long acquisition times. The F2T1 sequence has been proposed to acquire dGEMRIC datasets covering the entire hip joint with isotropic spatial resolution, which can then be reformatted during post-processing in rotating radial planes of the hip joint. These studies showed, for example, that dGEMRIC, images reformatted during post-processing in rotating radial planes can depict cartilage damage in the anterior-superior region of the acetabulum, where cartilage injury typically occurs in FAI patients.

These previously reported 3D dGEMRIC results were obtained, for example, at 1.5 Tesla with approximately 0.80 mm×0.80 mm×0.80 mm isotropic spatial resolution and acquisition times in the order of about 9-10 minutes or more, depending on the number of partitions needed to sample the whole 3D volume without aliasing artifacts. Given the small dimensions of hip acetabular cartilage, it may be preferable to further increase the spatial resolution, and reduce the scan time to minimize the loss in spatial resolution due to patient motion. One approach to increase the spatial resolution and/or reduce the scan time can be, for example, to perform 3D dGEMRIC at 3 Tesla and trade increased signal-to-noise ratio (SNR) for higher resolution and/or faster imaging (e.g., higher acceleration), respectively, at the expense of reduced accuracy due to increased B1+ variation within the hip at 3 Tesla. The loss in accuracy can be partially compensated with a corresponding B1+ mapping method, where the resulting flip angle maps can be used to correct the T₁ map.

Accordingly, it may be beneficial to address at least some of the issues and/or problems described herein above.

### SUMMARY OF EXEMPLARY EMBODIMENTS

These and other objects, features and advantages of the present disclosure will become apparent upon reading the following detailed description of exemplary embodiments of the present disclosure, when taken in conjunction with the appended drawings and claims.

According to exemplary embodiments of the present disclosure, apparatus, methods, and computer-accessible medium for generating a high-resolution 2D T₁ mapping sequence suitable for dGEMRIC in radial planes of the hip at 3 Tesla can be provided. The T₁ measurements can be accurate, repeatable and reproducible. An exemplary technique implemented by the exemplary apparatus, systems, methods, and computer-accessible medium can be applied to measure cartilage T₁ in other joints (e.g., knee, etc.) and T₁ of other tissues, and it can be suitable for applications at 3 Tesla, because it can be insensitive to B1+ inhomogeneities.

For example, according to certain exemplary embodiments of the present disclosure, it is possible to provide apparatus, methods, and computer-accessible medium for obtaining high spatial resolution 2D T₁ mapping. For example, an increased SNR facilitated by 3 Tesla imaging can be exploited by performing high spatial resolution 2D T₁ mapping in radial imaging planes to take advantage of the geometry of the hip joint (see, e.g., References 4 and 12). According to certain exemplary embodiments of the present disclosure, a B1-insensitive 2D T₁ mapping pulse sequence with high in-plane resolution for dGEMRIC in radial planes of the hip can be provided. Exemplary embodiments can, for example, image the hip using an exemplary fast spin-echo (FSE) pulse sequence at 3 Tesla to achieve high spatial resolution with adequate SNR and employ a B1-insensitive saturation pulse to perform uniform T₁ weighting. The scan time of the proposed pulse sequence can be, for example, about 1 minute and 20 second per 21) slice. Compared with the previously reported 3D dGEMRIC pulse sequence, the exemplary pulse sequence can be relatively less sensitive to patient motion. Further, according to certain exemplary embodiments of the present disclosure, the exemplary results can be validated, for example, against a rigorous multi-point saturation recovery (SR) pulse sequence at 3 Tesla, by comparing measured T₁ in a phantom and in the hip cartilage of FAI patients. Additionally, the accuracy and SNR efficiency of the exemplary pulse sequence against the 3D F2T1 pulse sequence can be compared in phantom experiments.

In certain exemplary embodiments of the present disclosure, it is possible to provide systems, methods and computer-accessible mediums for imaging at least one anatomical structure. For example, it is possible to direct a saturation-recovery (SR) pulse sequence having fast spin echo (FSE) to or at the anatomical structure(s). At least one T₁ image of the at least one anatomical structure can be generated based on the SR pulse sequence. According to certain exemplary embodiments, the anatomical structure(s) can include a hip. In certain exemplary embodiments, the T₁ image(s) can include a plurality of T₁ images generated or provided in a plurality of rotating radial planes.

According to certain exemplary embodiments, the SR pulse sequence can have a static magnetic field strength of greater than or equal to about 3 Tesla. In certain exemplary embodiments, the SR pulse sequence can include at least two image acquisitions. For example, the image acquisitions can include a proton-density (PD) acquisition and a T₁-weighted acquisition. According to certain exemplary embodiments, the SR pulse sequence can include a radio frequency (RF) saturation pulse. The RF saturation pulse can be substantially insensitive to an RF field (B₁) and/or static magnetic field (B₀) inhomogeneities.

These and other objects, features and advantages of the present disclosure will become apparent upon reading the following detailed description of exemplary embodiments of the present disclosure, when taken in conjunction with the appended drawings and claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is a block diagram of an exemplary role of a time delay (TD) according to a certain exemplary embodiment of the present disclosure;

FIG. 1B is a graph of an exemplary saturation recovery (SR) acquisition according to certain exemplary embodiments of the present disclosure;

FIG. 2 shows exemplary T₁ maps according to certain exemplary embodiments of the present disclosure;

FIG. 3 is a graph of exemplary T₁ measurements according to certain exemplary embodiments of the present disclosure;

FIG. 4 are exemplary images acquired using different time delay using apparatus, systems, methods, and computer-accessible medium according to certain exemplary embodiments of the present disclosure;

FIGS. 5A-5D are exemplary images of a hip generated using the apparatus, systems; methods, and computer-accessible medium according to certain exemplary embodiments of the present disclosure;

FIG. 6 are exemplary graphs of exemplary T₁ measurements compared to 6-point fitting according to certain exemplary embodiments of the present disclosure;

FIG. 7 are exemplary images of exemplary dGEMRIC T₁ maps generated using the apparatus, systems, methods, and computer-accessible medium according to certain exemplary embodiments of the present disclosure;

FIG. 8 is an illustration of an exemplary block diagram of an exemplary system in accordance with certain exemplary embodiments of the present disclosure; and

FIG. 9 is an exemplary flow diagram of an exemplary procedure, in accordance with certain exemplary embodiments of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures and indicated in appended claims.

### DETAILED DESCRIPTION OF EXEMPLARY EMBODIMENTS

### Exemplary Materials and Methods

Exemplary Pulse Sequence

With apparatus, systems, methods, and computer-accessible medium according to certain exemplary embodiments of the present disclosure, it is possible to provide, utilize and/or generate an exemplary FSE pulse sequence to perform two image acquisitions with two different T₁ weightings. The exemplary initial FSE image acquisition can be acquired, for example, after applying a saturation pulse with a SR time delay (TD) on the order of T₁ of the cartilage or other tissues of interest (e.g., accounting for the effect of gadolinium and magnetic field strength), in order to achieve a good balance between T₁ sensitivity and SNR for the SR acquisition (see, e.g., Haacke E, Brown R, Thompson M, Venkatesan R. Spin density, T1 and T2 quantification methods in MR imaging. Magnetic resonance imaging. New York: Wiley-Liss; 1999. p 637-667). Based on previous dGEMRIC studies at 1.5 Tesla and 3 Tesla, T₁ of normal cartilage at 3 Tesla can be expected to be, for example, on the order of about 700-800 ms. As such, TD 700 ms can be used, for example, to achieve a good balance between T₁ sensitivity and SNR for the SR acquisition. In the exemplary SR acquisition with TD=700 ms, tissues with short T₁ values (e.g., <350 ms) can be susceptible to random error, due to near complete recovery of magnetization, whereas tissues with long T₁ values (e.g., >2100 ms) can be susceptible to random error, due to insufficient recovery of magnetization. The second exemplary FSE image (e.g., proton density-weighted (PD)) acquisition can be performed with repetition time (TR) on the order of, for example, 5 T₁s and without the saturation pulse. T₁ can be calculated pixel-wise, for example, by dividing the SR image, I_{SR}, by the PD image, I_{PD}, to correct for the unknown equilibrium magnetization (M₀), and then solving the ideal SR experiment described by the Bloch equation governing T₁ relaxation, e.g.:

$$\begin{matrix}\frac{{I}_{SR} = {M}_{0} ( 1 - {e}^{- TD / {T}_{1}} )}{{I}_{PD} = {M}_{0}} = ( 1 - {e}^{- TD / {T}_{1}} ) , & [ 1 ] \\ {T}_{1} = \frac{- TD}{log ( 1 - \frac{{I}_{SR}}{{I}_{PD}} )} & [ 2 ]\end{matrix}$$

For example, the apparatus, systems, methods, and computer-accessible medium according to certain exemplary embodiments of the present disclosure can implement the exemplary FSE pulse sequence on a whole-body 3 Tesla MRI scanner (e.g. Verio, by Siemens Healthcare, Erlangen, Germany) equipped with a gradient system capable of achieving a maximum gradient strength of, e.g. 45 mT/m and a slew rate of 200 T/m/s. The radio-frequency (RF) excitation can be performed using a transmit body coil, and a 32-element “cardiac” coil array (e.g., by Invivo, Orlando, Fla.) can be employed for signal reception. The relevant imaging parameters can include, e.g.: field of view=190 mm×190 mm; acquisition matrix=320×320; in-plane resolution=0.6 mm×0.6 mm; slice thickness 5 mm; turbo factor=13; FSE readout duration can be, for example, about 143 ms, TE=10 ms, refocusing flip angle can be, for example, 180°, generalized auto-calibrating partially parallel acquisitions (GRAPPA) with an acceleration factor=1.8, and receiver bandwidth=161 Hz/pixel. A fat suppression pulse can be used to avoid chemical shift artifacts at the bone-cartilage interface. TR (e.g., including the saturation pulse, recovery time, and FSE readout duration) can be 850 ms and 4000 ms for SR and PD acquisitions, respectively. Total scan time for both SR and PD acquisitions can be, for example, about 1 min. 20 sec. per slice.

The apparatus, systems, methods, and computer-accessible medium according to certain exemplary embodiments of the present disclosure can also provide, utilize and/or generate a B₁-insensitive saturation pulse to achieve uniform T₁ weighting within the hip at 3 Tesla. The hybrid adiabatic-rectangular pulse train can include three non-selective RF pukes, non-selective rectangular 140° pulse, non-selective rectangular 90° pulse, and non-selective adiabatic half-passage pulse. The crusher gradients inserted between RF pulses can be cycled to eliminate stimulated echoes. Spoiler gradients can be applied before the first RF pulse and after the third RF pulse to dephase the transverse magnetization.

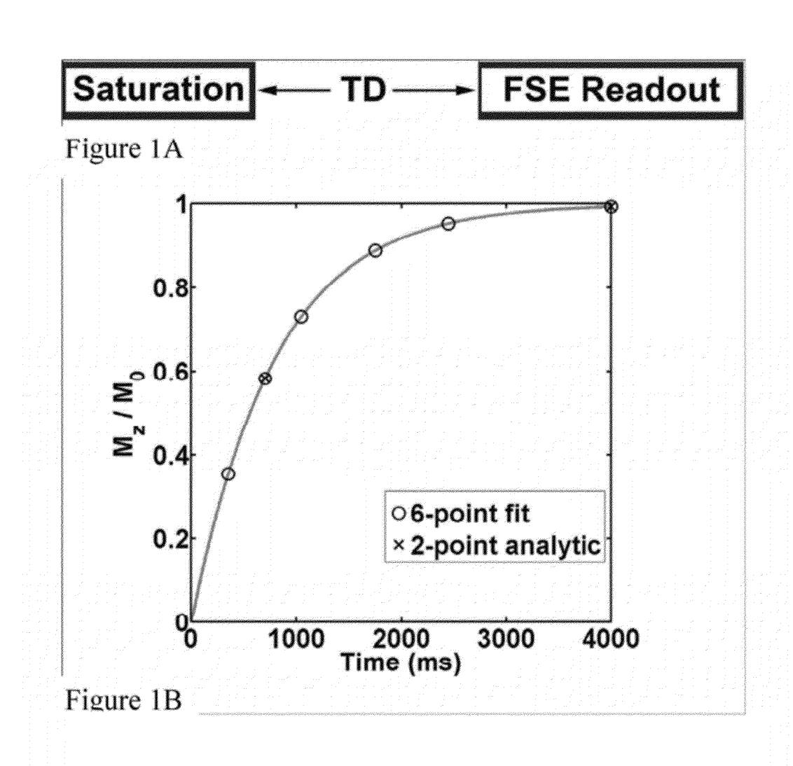

In order to validate the exemplary T₁ measurements calculated and/or determined with exemplary Equation [2], four additional SR images can be acquired, for example, with TD=350, 1.050, 1750, 2450 ms (see, e.g., FIGS. 1A and 1B). Total scan time for the 4 additional SR images can be, for example, 1 min 40s per slice. These additional SR images can be combined with the exemplary SR image with TD=700 ms and the exemplary PD image, in order to perform a two-parameter (e.g., M₀, T₁) non-linear fit of exemplary Equation [1]. The six exemplary images can be acquired in series to minimize image registration errors. Total scan time to acquire the six images can be for example, 3 min per slice. FIG. 1B shows an exemplary graph of exemplary SR acquisitions which can be used in and/or with one or more exemplary embodiments of the present disclosure. For example, five SR acquisitions are shown with TDs 350 ms, 700 ms, 1050 ms, 1750 ms, and 2450 ms. The exemplary PD acquisition can be obtained with TR=4000 ms and without the saturation pulse. The exemplary analytical T1 measurement can be made using the SR image with TD=700 ms and PD image (e.g., see Equation 1). The exemplary two-parameter fit of the ideal SR equation can be made using all six exemplary images. Further, e.g., all six images can be acquired in series, in order to minimize image registration errors.

Certain exemplary experiments can be performed to verify certain exemplary embodiments of the present disclosure. For example, the exemplary 2D FSE pulse sequence can be compared against the 3D F2T1 pulse sequence, for example, in two exemplary phantom experiments. In the first exemplary phantom experiment designed to compare, for example, their sensitivity to B1+ variations, an exemplary 2D T₁ mapping pulse sequence was performed with the exemplary protocol, and 3D F2T1 imaging was performed with the following parameters, e.g.: spatial resolution=0.8 mm×0.8 mm×0.8 mm, flip angles=5° and 30°, TE/TR=3.5/20 ins, receiver bandwidth=130 Hz/pixel, 144 partitions, 22% partition over sampling, 41% partition over sampling, GRAPPA acceleration factor=1.8, partial Fourier factor 6/8 in the phase-encoding direction, and scan time=13 min 16s. Prior to the 3D F2T1 sequence, a B₁+ mapping prescan, based on a stimulated echo pulse sequence, was performed, for example, to correct the T₁ maps calculated from the 3D F2T1 images. The T₁ maps with B₁+ correction were computed, for example, using the Siemens inline reconstruction procedure on an exemplary 3 Tesla scanner equipped with, e.g., VB 17 software platform. For the second exemplary phantom experiment designed to compare their SNR efficiencies, both the exemplary 2D T₁ mapping and 3D F2T1 mapping procedures were performed, for example, with full k-space encoding (e.g., no GRAPPA acceleration and no partial Fourier imaging), where the scan time was, for example, about 2 minutes and 15 seconds and 31 minutes and 48 seconds, respectively, in order to calculate the SNR as the ratio of the mean signal and standard deviation of background noise.

Exemplary Phantom Imaging

A spherical mineral oil phantom with a known T₁ (e.g., ˜550 ms) in the coronal plane can be imaged, for example, to determine the sensitivity of the saturation pulse to clinically relevant B₁+ variations within the hip at 3 Tesla. To avoid signal saturation of the oil phantom, the exemplary phantom experiment can be performed, for example, without the fat suppression pulse. Image acquisition can be repeated, for example, with B₁+ scale of the saturation pulse manually adjusted from about 0.8-1.2 (e.g., 0.1 steps) of its nominally calibrated B₁+ value. Nominal B₁+ can be determined, for example, using the automated RF transmit calibration procedure. The upper limit of 20% B₁+ variation can be based on preliminary experience with hip imaging at 3 Tesla.

In a second exemplary experiment, the phantom can include, e.g., approximately 9% glycerol in distilled water to emulate relaxation times of hip cartilage (e.g., measured T₁=730 ms; measured T2=37 ms). For the 3D data, SNR was measured, for example, only in a 2D plane that typically corresponds to the 2D FSE plane. To account for the difference in voxel sizes, the SNR were normalized by the voxel size. The exemplary normalized SNR efficiency was then determined as the normalized SNR divided by the square root of the scan time.

Exemplary Hip Imaging

In the exemplary experiments, patients with hip pain and positive physical examination for FAI were imaged after a double dose (e.g., 0.2 mmol/kg) intravenous injection of Gd-DTPA²⁻ (e.g., Magnevist®, by Bayer Healthcare) and 15 minutes walking on a treadmill at controlled speed. The dGEMRIC pulse sequence was applied, for example, after the clinical protocol, approximately 45 minutes after administration of Gd-DTPA. Ten hips (e.g., 6 left, 4 right) were scanned in nine consecutive patients (e.g., mean age=36±10 years). The images were acquired in a radial plane that included the anterior-superior region of the acetabulum. Human imaging was performed in accordance with protocols approved by the Human Investigation Committee; and the subjects provided written informed consent.

Exemplary Image Analysis

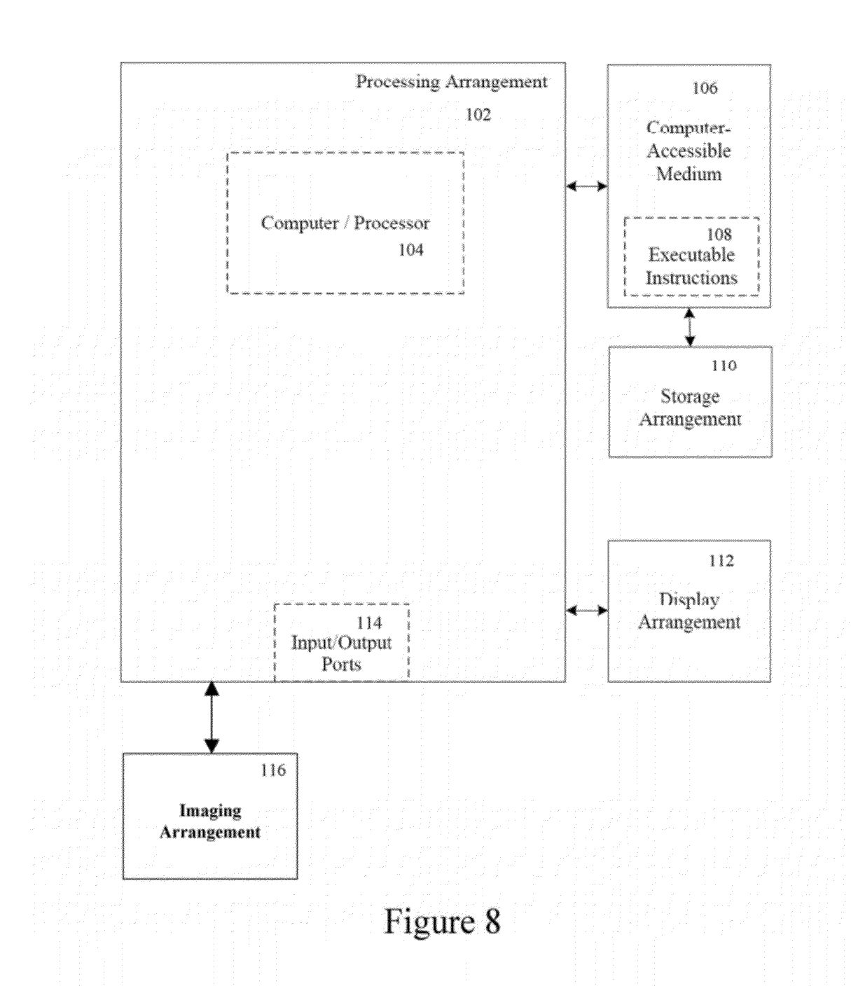

Image processing can be performed, for example, using an exemplary software in accordance with the exemplary embodiments of the present disclosure, which can be implemented by an exemplary system shown in FIG. 8. For each hip, the six images acquired at different time points (see FIG. 1B) were, for example, spatially registered to the PD image to compensate for motion. In particular, affine transformation was used, for example, to register a user-defined ROI preferably including the entire hip joint.

After de-identification and randomization of the patient data, two observers, for example, manually segmented a region of interest (ROI) over the weight-bearing portion of the hip articular cartilage (see, e.g., Mamisch T C, Dudda M, Hughes T, Burstein D, Kim Y J. Comparison of delayed gadolinium enhanced MRI of cartilage (dGEMRIC) using inversion recovery and fast T1 mapping sequences. Magnetic Resonance in Medicine 2008; 60(4):768-773), extending from the lateral bony edge, not including the labrum, to the edge of the acetabular fossa, For each ROI, the exemplary software calculated an exemplary solved T1 map based on the formula in exemplary Equation [2] (e.g., using TD=700 ms and PD). As a reference measurement, the exemplary software also calculated a two-parameter six-point fitted T₁ map based on exemplary Equation [1], using six images and a global optimization procedure (see, e.g., Hansen E, Walster G. Global optimizing using interval analysis: revised and expanded. New York: Marcel Dekker, Inc; 2003). Observer 1 repeated the image analysis, for example, after 14 days from the first analysis to assess intra-observer variability. Inter-observer variability was assessed, for example, between observer 1 and observer 2, comparing the average T₁ value in the cartilage ROI for each hip. The two independent observers were blinded to patient identity and each other.

### Statistical Analysis

For each ROI, the difference between the exemplary T₁ and the six-point fit T₁ was calculated, for example, pixel-wise in order to display the spatial distribution of error for each analysis session. The Pearson correlation and Bland-Altman (see, e.g Bland J M, Altman DG. Statistical methods for assessing agreement between two methods of clinical measurement. Lancet 1986; 1:307-310) analyses were performed, for example, using the mean T₁ value in each ROI.

### Noise Analysis

To estimate the T₁ error, a theoretical analysis can be performed, for example, using exemplary Equation ill for reference T₁ mapping (e.g., 6-point SR experiment) and exemplary Equation [2] for exemplary T₁ mapping, as a function of true T₁ ranging from 600 to 1200 ms (e.g., 5 ms steps). The lower (e.g., normal−200 ms) and upper (e.g., normal+400 ms) limits of the T₁ range can be based, for example, on assuming normal cartilage T₁ equal to 800 ms. For example, to estimate clinically relevant white Gaussian noise, in a 27-years-old male volunteer, two PD image acquisitions can be acquired in radial planes of the hip with full k-space encoding and TR=10 s (e.g., >5T1). In addition, a noise map can be acquired, for example, using the same pulse sequence without RF excitation. The hip articular cartilage can be segmented manually, and the SNR can be calculated as the ratio of the mean cartilage signal and standard deviation of noise derived from the noise map. The average of two PD SNR measurements can be, e.g., 127.5. Given that the exemplary PD acquisition can perform GRAPPA acceleration 1.8, a PD SNR of 95 can be anticipated. Assuming M₀ PD, clinically relevant white Gaussian noise was estimated as, e.g., 0.0105M₀ (e.g., =M₀/95). The theoretical noise analysis can be repeatedly performed, for example, 100 times using a numerical phantom with 100 pixels to mimic the typical number of pixels in the segmented hip cartilage, where identical amount of white noise was added, for example, to the numerical PD and SR images. The influence of white noise on T₁ accuracy can be estimated, for example, by performing linear regression analysis on the calculated and true T₁ values and calculating root-mean-square-error (RMSE). Reported linear regression statistics and RMSE values represent the mean standard deviation over 100 measurements.

### EXEMPLARY RESULTS

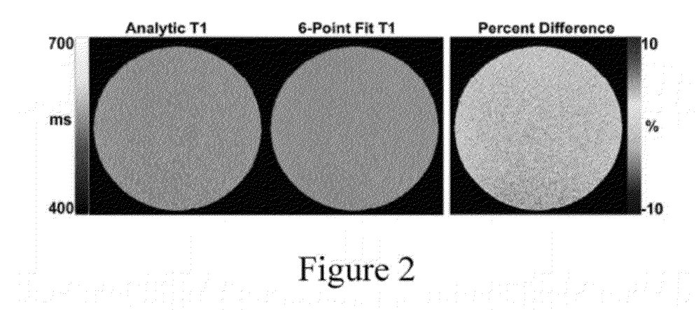

FIG. 2 shows exemplary maps of the phantom obtained using certain exemplary embodiments of the present disclosure and six-point T₁ method, as well as the percentage difference map. T₁ maps were calculated in FIG. 2 using the exemplary 6-point fit method/procedure for a spherical mineral oil phantom with a known T₁ (e.g., ˜550 ms). The exemplary phantom was imaged on a coronal plane, e.g., without the fat suppression pulse. The difference between the two T₁ maps was determined pixel-wise, e.g., for the entire phantom. T₁ in the phantom was, e.g., 562±21 ms with the exemplary method and, e.g., 561±15 ms with the six-point fit method, and RMS of percent difference was 2.8%, suggesting that they are quantitatively equivalent. T₁ measurements with the exemplary method were, e.g., 567 ms, 565 ms, 561 ms, 561 ms, and 563 ms for B₁+ scales 0.8, 0.9, 1.0, and 1.1, and 1.2, respectively. Consistent with work in the heart at 3 Tesla (see, e.g., Reference 20), the phantom T₁ values were similar throughout (e.g., less than 1% difference with respect to the average value), suggesting that the saturation pulse can be insensitive to B₁+ variation as large as 20%.

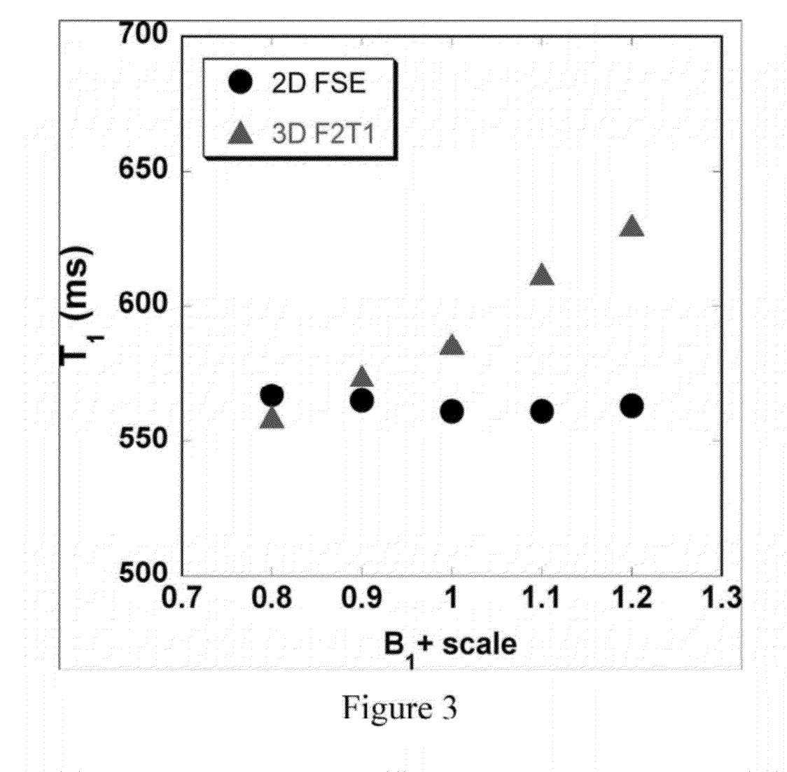

In contrast, T₁ measurements using the 3D F2T1 pulse sequence with B₁+ correction were, e.g., 559 ms, 574 ms, 585 ms, 612 ms, and 630 ms for B₁+ scales, e.g., 0.8, 0.9, 1.0, and 1.1, and 1.2, respectively, indicating that even with B₁+ correction the 3D F2T1 pulse sequence can be sensitive to clinically relevant B₁+ variation (see FIG. 3). FIG. 3 shows an exemplary graph of T₁ measurements as a function of B₁+ scale ranging from 0.8 to 1.2 (0.1 steps). The 3D F2T1 pulse sequence can be sensitive to B₁, scale ranging from 0.8 to 1.2, whereas an exemplary proposed 2D T₁ mapping pulse sequence can be insensitive to the same B1+ scale range.

For the exemplary glycerol phantom experiment, the normalized SNR efficiency was, for example, about 10.3 and 4.3 for the 2D FSE and 3D F2T1, respectively. The higher SNR efficiency of 2D FSE over 3D F2T1 can be due to the difference in flip angles (e.g., 90-180° vs. 5-30°; 2D FSE vs. 3D F2T1, respectively).

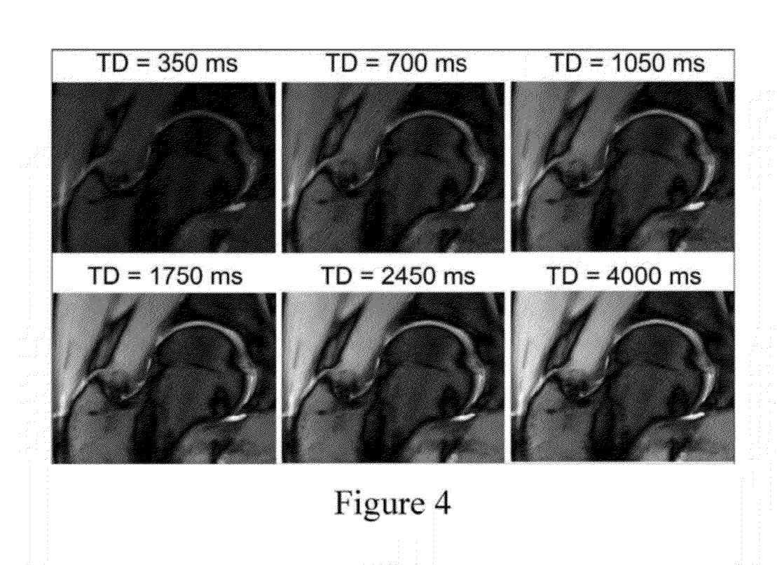

FIG. 4 shows, for one representative case, six exemplary radial images acquired with different SR time delays. T₁ was calculated rigorously by, e.g., fitting the saturation recovery (SR) curve with the signals of the six images. T₁ was also calculated with the analytic formula in exemplary Equation 1 using, e.g., the second and last image. TD values were selected assuming T₁ in the order of 700-800 ms in healthy hip cartilage at 3 Tesla, so that the image at TD=4 s corresponds to proton density. Further, this exemplary image series exhibits consistently good image quality. For pixels within the ROIs, global optimization using the six available values can allow an accurate fitting of the SR curve, e.g., as shown in FIG. 1B to calculate T₁.

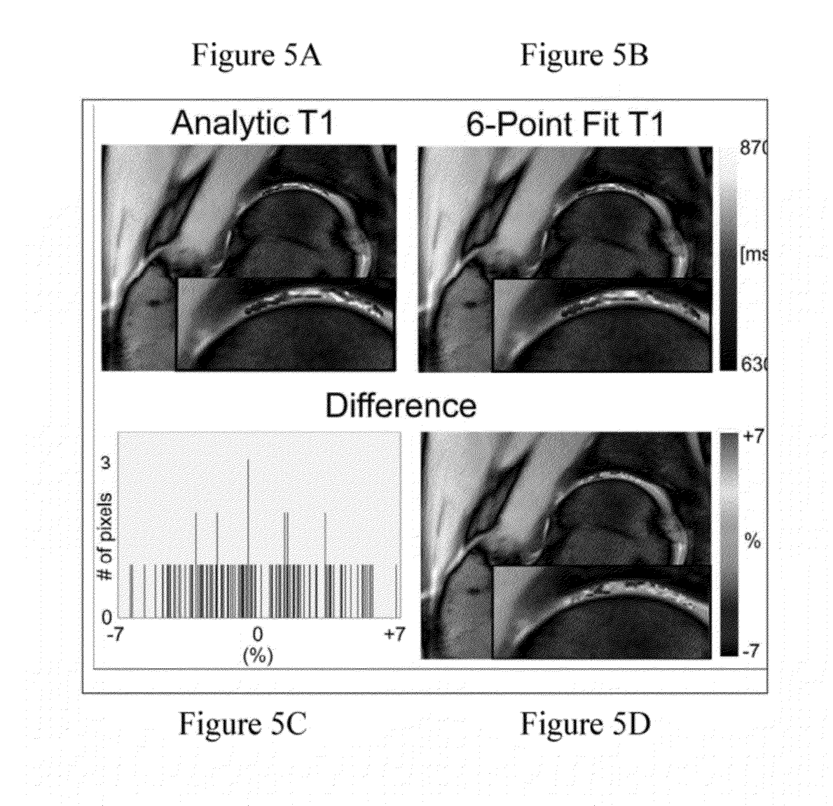

Exemplary and six-point fit T₁ maps are shown, for example, for one hip in FIG. 5, together with a map and a histogram of the percent difference between the two. For one, some or all cases, the weight-bearing portion of hip cartilage can be segmented from the lateral bony edge to the edge of the acetabular fossa. T₁ maps can be determined using the exemplary and the 6-point fit methods/procedures for each ROI (e.g., as illustrated in FIGS. 5A and 5B) and the percent difference between the two ROIs was determined pixel-wise (e.g., as illustrated in FIGS. 5C and 5D). The RMS of percent difference was 3.2% for the hip in this figure.

The range of the color bars were chosen, for example, to span the distribution of values in the ROIs. In this particular hip, the pixel-wise percent difference between analytic and six-point fit T₁ ranged from, for example, −6.4 to 6.8%, and the RMS of percent difference was 3.2.

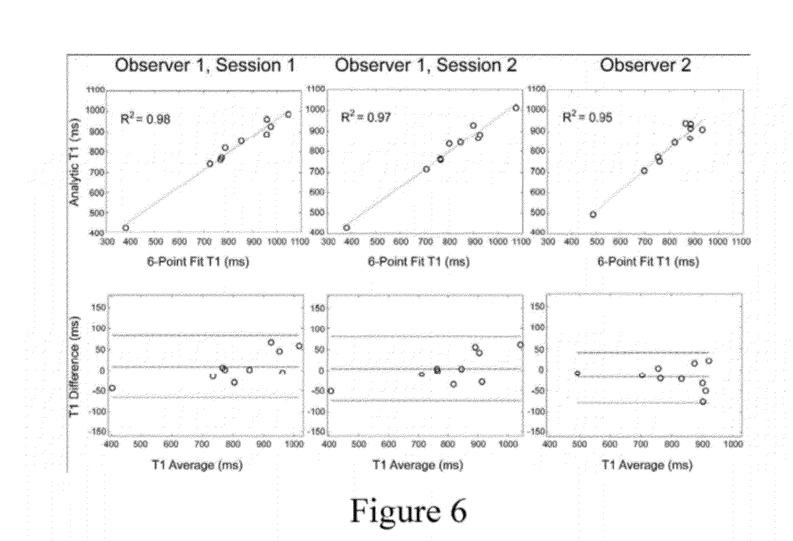

The mean T₁ over 10 hips was, for example, 823±189 ms, 808±183 ms and 797±132 ms, for the two sessions of observer 1 and the single session of observer 2, respectively. The fact that mean T₁ of cartilage was on the order of 800 ms can confirm the choice in TD of 700 ms. The top row of FIG. 6 shows, for example, the correlation between exemplary and six-point fit T₁ for the ten hips, whereas the bottom row shows Bland-Altman plots that can illustrate the agreement between the two T₁ measurements. The Person correlation coefficient of determination R² can be larger than 0.95 in all cases (e.g., p<0.001), suggesting that the two measurements can be strongly correlated. According to the Bland-Altman analysis, exemplary six-point fit T₁ values were in good agreement (e.g., mean difference=−8.7 ms, e.g., ˜1%; upper and lower 95% limits of agreement=64.5 and −81.9 ms, respectively). Pearson and Bland-Altman statistics for observer 1, analysis 2 and observer 2 are shown in Table 1.

TABLE I
SUMMARY OF BLAND-ALTMAN AND PEARSON ANALYSIS.

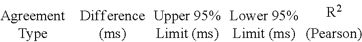

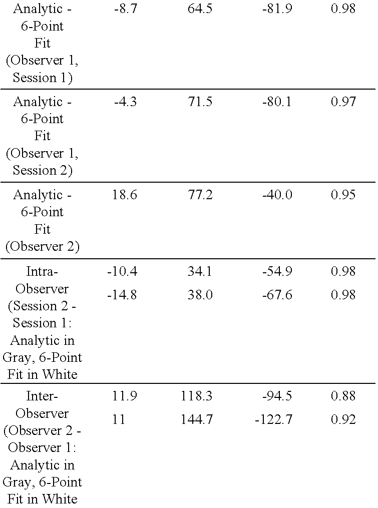

As summarized in Table 1, the intra-/inter-observer variability in T₁ calculated from the same SR data with the analytic method can be, e.g., −10.4/11.9 ms, and the upper (e.g., mean plus 1.96 standard deviation) and lower (e.g., mean minus 1.96 standard deviation) 95% limits of agreement were 34.1/118.3 ms and −54.9/94.5 ms, respectively. Using the six-point fit, the intra-/inter-observer variability in T₁ can be −14.8/11 ins, whereas the upper and lower 95% limits of agreement can be 38.0/144.7 ms and −67.6/122.7 ms, respectively.

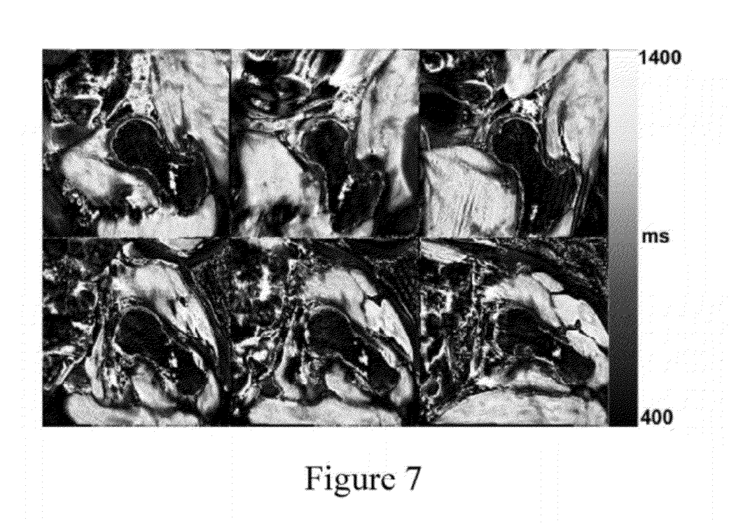

FIG. 7 shows, for example, exemplary representative dGEMRIC T₁ maps of a 53-year-old male patient in six rotating radial planes of the hip joint. The total scan time to acquire the six T₁ maps was, in this exemplary embodiment, e.g., 8 min. Both raw SR and PD images exhibited good image quality, and these T₁ maps depict the hip cartilage with adequate spatial resolution.

For the theoretical noise analysis, RMSE values were, for example, 27.3±1.6 and 20.3±1.6 ms for the analytic and 6-point fit T₁, respectively, compared with true T₁ ranging from 600 to 1200 ms. Linear regression statistics were comparable between the analytic and 6-point T₁ mapping methods (see Table 2).

TABLE 2

| Measurement | | | | |
|---|---|---|---|---|
| Pair | Slope | Bias (ms) | R² | RMSE (ms) |
| Analytic T1 vs. | 1.01 ± 0.01 | 9.60 ± 9.08 | 0.99 ± 0.00 | 27.3 ± 1.6 |
| True T1 | | | | |
| 6-Point Fit T1 | 1.00 ± 0.01 | 0.35 ± 9.26 | 0.99 ± 0.00 | 20.3 ± 1.6 |
| vs. True T1 | | | | |

### FURTHER DETAILED DESCRIPTION OF EXEMPLARY EMBODIMENTS

The apparatus, systems, methods, and computer-accessible medium according to exemplary embodiments of the present disclosure can provide, utilize and/or generate a two-dimensional (2D) T₁ mapping pulse sequence for dGEMRIC in the hip joint with a clinically acceptable scan time of, e.g., 1 min 20 seconds per slice. Compared with a rigorous six-point SR acquisition (e.g., 3 min per slice), the exemplary T₁ mapping acquisition using the exemplary procedure according to the exemplary embodiments of the present disclosure can produce accurate results in vitro and in vivo, suggesting that the two acquisitions can be quantitatively equivalent. The intra- and inter-observer agreements for T₁ calculations can be good.

Conventional 2D T₁ mapping pulse sequences based on multi-point IR or SR with FSE readout (see, e.g., Crawley A P, Henkelman R M. A comparison of one-shot and recovery methods in T1 imaging. Magnetic Resonance in Medicine 1988; 7(1):23-34; see also, Haase A. Snapshot FLASH MRI. Applications to T1, T2, and chemical-shift imaging. Magnetic Resonance in Medicine 1990; 13(1):77-89; see also Look Locker D. Time saving in measurement of NMR and EPR relaxation times. Rev Sci Instrum 1970; 41:250-251) are likely clinically not feasible due to their long acquisition times. T₁ mapping pulse sequences based on gradient echo readout (see, e.g. References 8, 27) can be more efficient than FSE based pulse sequences, but they can be generally low in SNR and sensitive to B1+ inhomogeneities at 3 Tesla. The exemplary 2D pulse sequence according to certain exemplary embodiments of the present disclosure can provide good image quality, because, e.g., FSE readout at 3 Tesla can be used. Furthermore, such exemplary pulse sequence can facilitate a uniform T₁ weighting by utilizing a robust saturation pulse (see, e.g., Kim D, Oesingmann N, McGorty K. Hybrid adiabatic-rectangular pulse train for effective saturation of magnetization within the whole heart at 3 T. Magnetic Resonance in Medicine 2009; 62(6):1368-1378). This exemplary saturation pulse can effectively saturate the magnetization within the whole heart at 3 Tesla (see, e.g., Id.). B1+ variation can be lower within the hip than within the heart. The exemplary phantom experiments indicated that, compared with 3D F2 T₁ pulse sequence, for example, the exemplary proposed 2D T₁ mapping pulse sequence can yield higher SNR efficiency and lower sensitivity to B1+ variations. The exemplary phantom experiment were performed assuming B1+ variation as large as 20%, based on preliminary experience with hip imaging at 3 Tesla. The exemplary T₁ mapping pulse sequence can be insensitive to up to 40% B1+ variation (see, e.g., Id.).

The exemplary pulse sequence can be validated, for example, against a rigorous exemplary T₁ mapping method based on a six-point SR acquisition. A potential issue with this acquisition approach in-vivo can be patient motion. While an affine transformation was used, for example, to perform image registration of the entire hip joint, there was small residual motion between images which could have affected T₁ calculation for some of the pixels. The motion is likely to be less of an issue for the two-point SR acquisition of 1 minute and 20 seconds than the full six-point SR acquisition of 3 min. An exemplary approach to further minimize the registration error can be to perform interleaved acquisition between SR and PD.

The mean T₁ of cartilage can be, for example, on the order of 800 ms. As such, the exemplary choice TD=700 ms for the SR image acquisition can represent a good balance between T₁ sensitivity and SNR, and TR=4000 ms for the PD image acquisition can be sufficient. For imaging tissues with different T₁, both TD for SR and TR for PD acquisitions are preferably adjusted.

FIG. 8 shows an exemplary block diagram of an exemplary embodiment of a system according to the present disclosure. For example, exemplary procedures in accordance with the present disclosure described herein can be performed by a processing arrangement and/or a computing arrangement 102. Such processing/computing arrangement 102 can be, e.g., entirely or a part of, or include, but not limited to, a computer/processor 104 that can include, e.g., one or more microprocessors, and use instructions stored on a computer-accessible medium (e.g., RAM, ROM, hard drive, or other storage device).

As shown in FIG. 8, e.g., a computer-accessible medium 106 (e.g., as described herein above, a storage device such as a hard disk, floppy disk, memory stick, CD-ROM, RAM, ROM, etc., or a collection thereof) can be provided (e.g., in communication with the processing arrangement 102). The computer-accessible medium 106 can contain executable instructions 108 thereon. In addition or alternatively, a storage arrangement 110 can be provided separately from the computer-accessible medium 106, which can provide the instructions to the processing arrangement 102 so as to configure the processing arrangement to execute certain exemplary procedures, processes and methods, as described herein above, for example.

Further, the exemplary processing arrangement 102 can be provided with or include an input/output arrangement 114, which can include, e.g., a wired network, a wireless network, the internet, an intranet, a data collection probe, a sensor, etc. As shown in FIG. 8, the exemplary processing arrangement 102 can be in communication with an exemplary display arrangement 112, which, according to certain exemplary embodiments of the present disclosure, can be a touch-screen configured for inputting information to the processing arrangement in addition to outputting information from the processing arrangement, for example. Further, the exemplary display 112 and/or a storage arrangement 110 can be used to display and/or store data in a user-accessible format and/or user-readable format.

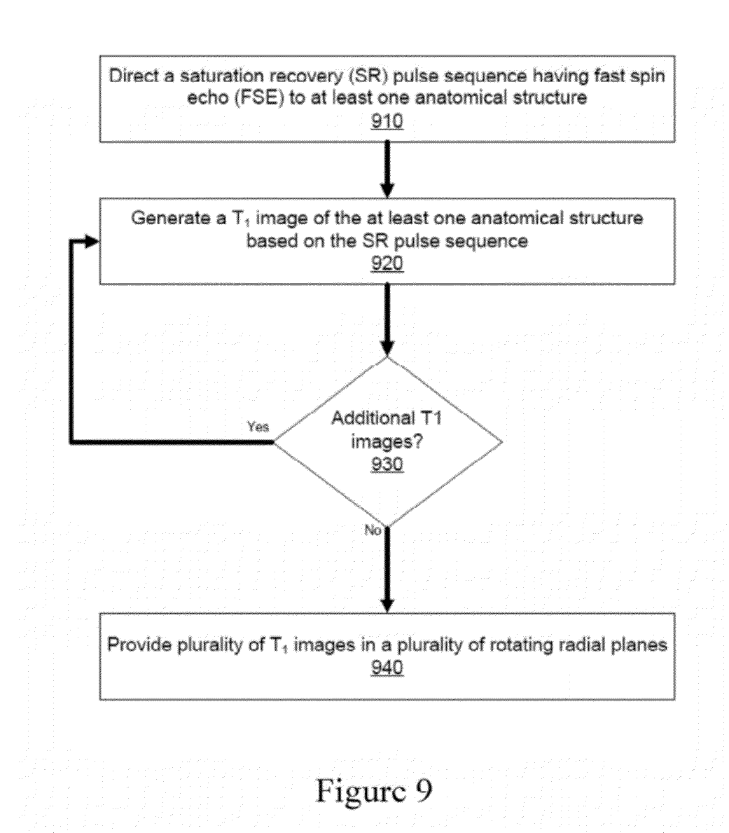

FIG. 9 illustrates an exemplary flow of an exemplary procedure, according to one or more exemplary embodiments of the present disclosure. For example, at block 910, the exemplary procedure can direct a saturation recovery (SR) pulse sequence having fast spin echo (FSE) to at least one anatomical structure (e.g., a hip). Next, at block 920, the exemplary procedure can generate at least one T₁ image of the at least one anatomical structure based on the SR pulse sequence. The exemplary procedure can generate one image, or a plurality of images via block 930. Additionally, in certain exemplary embodiments, it is possible to provide at least one (e.g. a single or a plurality) of T₁ images in a plurality of rotating radial planes, e.g., at block 940.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. For example, various exemplary embodiments described herein can be used interchangeably, in conjunction and together with other exemplary embodiments of the present disclosure. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for imaging at least one anatomical structure, comprising:
directing a saturation recovery (SR) pulse sequence having fast spin echo (FSE) to or at the at least one anatomical structure; and
generating at least one T₁ image of the at least one anatomical structure based on the SR pulse sequence.

2. The method of claim 1, wherein the at least one anatomical structure includes a hip.

3. The method of claim 2, wherein the at least one T₁ image includes a plurality of T₁ images generated or provided in a plurality of rotating radial planes.

4. The method of claim 1, wherein the SR pulse sequence has a static magnetic field strength of greater than or equal to about 3 Tesla.

5. The method of claim 1, wherein the SR pulse sequence includes at least two image acquisitions.

6. The method of claim 5, wherein the image acquisitions include a proton-density (PD) acquisition and a T₁ weighted acquisition.

7. The method of claim 6, wherein the SR pulse sequence includes a radio frequency (RF) saturation pulse.

8. The method of claim 7, wherein the RF saturation pulse is substantially insensitive to at least one of an RF field (B₁) or static magnetic field (B₀) inhomogeneities.

9. A non-transitory computer readable medium for imaging at least one anatomical structure including instructions thereon that are accessible by a hardware processing arrangement, wherein, when the processing arrangement executes the instructions, the processing arrangement is configured to:
direct a saturation-recovery (SR) pulse sequence having fast spin echo (FSE) at the at least one anatomical structure; and
generate at least one T₁ image of the at least one anatomical structure based on the SR pulse sequence.

10. The computer readable medium of claim 9, wherein the at least one anatomical structure includes a hip.

11. The computer readable medium of claim 10, wherein the at least one T₁ image includes a plurality of T₁ images generated or provided in a plurality of rotating radial planes.

12. The computer readable medium of claim 9, wherein the SR pulse sequence has a static magnetic field strength of greater than or equal to about 3 Tesla.

13. The computer readable medium of claim 9, wherein the SR pulse sequence includes at least two image acquisitions.

14. The computer readable medium of claim 13, wherein the image acquisitions include a proton-density (PD) acquisition and a T₁-weighted acquisition.

15. The computer readable medium of claim 14, wherein the SR pulse sequence includes a radio frequency (RF) saturation pulse.

16. The computer readable medium of claim 15, wherein the RF saturation pulse is substantially insensitive to at least one of an RF field (B₁) or static magnetic field (B₀) inhomogeneities.

17. A system for imaging at least one anatomical structure, comprising:
a non-transitory computer readable medium including instructions thereon that are accessible by a hardware processing arrangement, wherein, when the processing arrangement executes the instructions, the processing arrangement is configured to:
a. direct a saturation-recovery (SR) pulse sequence having fast spin echo (FSE) at the at least one anatomical structure; and
b. generate at least one T₁ image of the at least one anatomical structure based on the SR puke sequence.

18. The system of claim 17, wherein the at least one anatomical structure includes a hip.

19. The system of claim 18, wherein the at least one T₁ image includes a plurality of T₁ images generated or provided in a plurality of rotating radial planes.

20. The system of claim 17, wherein the SR, pulse sequence has a static magnetic field strength of greater than or equal to about 3 Tesla.

21. The system of claim 17, wherein the SR pulse sequence includes at least two image acquisitions.

22. The system of claim 21, wherein the image acquisitions include a proton-density (PD) acquisition and a T₁-weighted acquisition.

23. The system of claim 22, wherein the SR pulse sequence includes a radio frequency (RF) saturation pulse.

24. The system of claim 23, wherein the RF saturation pulse is substantially insensitive to at least one of an RF field (B₁) or static magnetic field (B₀) inhomogeneities.
